(19) United States
(12) Patent Application Publication
(10) Pub. No.: US 2009/0017080 A1
(43) Pub. Date: Jan. 15, 2009

(54) PERSONAL CARE KIT HAVING SKIN CARE COMPOSITIONS WITH A READILY PERCEPTIBLE DIFFERENCE

(76) Inventors: Paul Robert Tanner, Lebanon, OH (US); Larry Richard Robinson, Loveland, OH (US); Robert Bao Ha, Liberty, OH (US); Dean Arthur Zimmerman, West Chester, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/047,391

(22) Filed: Mar. 13, 2008

Related U.S. Application Data
(60) Provisional application No. 60/918,216, filed on Mar. 15, 2007.

(51) Int. Cl. A61K 8/02; A61Q 19/00

(52) U.S. Cl. 424/401

(57) ABSTRACT

A personal care kit comprises a first series of unit-dose packages containing a first skin care composition and a second series of unit-doses packages containing a second skin care composition, wherein the first skin care composition and the second skin care composition exhibit a readily perceptible difference. The readily perceptible difference may be a visible difference, a tactile difference, or an olfactory difference. The first and/or second skin care composition may comprise a skin care active. A method for using the personal care kit is also disclosed.

### CROSS REFERENCE TO RELATED APPLICATION

This application claims the benefit under 35 USC 119(e) to U.S. Application Ser. No. 60/918,216, filed Mar. 15, 2007.

### FIELD OF THE INVENTION

The present invention relates to a personal care kit having a first series of unit-dose packages containing a first skin care composition and a second series of unit-doses packages containing a second skin care composition. The first skin care composition and the second skin care composition exhibit a readily perceptible difference.

### BACKGROUND OF THE INVENTION

Consumers are faced with an overwhelming number of skin care compositions within the marketplace. These compositions may comprise skin care actives that provide a benefit through regulating and/or improving skin condition. There are numerous skin care actives, as are well known in the art, directed to a variety purposes such as addressing the sign of skin aging or treating insult-affected skin and other keratinous tissue. While some skin care actives exhibit acute benefits that are immediately appreciable to a user, many skin care actives exhibit chronic benefits that are realized after a period of time. Usually the chronic benefit is achieved by routine application of the skin care composition over several days, weeks, or months. Unfortunately, many users do not adhere to a routine application of the skin care composition that is necessary to achieve the chronic benefit. For example, after a week of daily application of a skin care composition, some users may become discouraged that a chronic benefit is not visibly or tactily perceptible. Other users may simply lose interest in regular application of the skin care composition. This is particularly true if the skin care composition is directed to regulating skin condition such that a user's skin is maintained without degradation. In such a case, the user can not appreciate that the skin care composition is providing a chronic benefit since the maintenance benefit may only be appreciated by discontinuing use and then suffering a degradation in skin condition.

Another issue related to skin care compositions that exhibit chronic benefits is the issue of dosing. Many skin care compositions are provided to the user in a bulk package suitable for several applications. Users often do not apply the idea dose. By applying too little of the skin care composition, the chronic effect may be delayed or may never materialize. The user may become discouraged with the skin care composition even though the composition is effective when dosed properly. Conversely, by applying too much of the skin care composition, materialization of the chronic effect is not accelerated. The user may become discouraged with quantity and cost of skin care composition being used even though the composition is beneficial and cost effect when dosed properly. In both cases, incorrect dosing of skin care composition from a bulk package can impact the chronic benefit perceived by the user. Further complicating the use of bulk packages is variable dosing. In some situations, it is necessary to vary the dose of skin care composition. For example, the amount of skin care composition may be gradually increased or decreased during the course of a dosing regimen. User compliance to variable dosing is poor when using a bulk package.

In light of the discussion provided above, there exists a need in the personal care industry and, more specifically, the skin care industry for an easy to use personal care kit comprising a plurality of skin care compositions in unit-dose packaging to address, at least in part, the issues of improper dosing. A need also exists for the personal care kit to comprise a plurality of skin care compositions that exhibit a readily perceptible difference to address, at least in part, the issue surrounding what may be a prolonged appearance of a chronic benefit or the lack of appreciation of the chronic benefit.

### SUMMARY OF THE INVENTION

In light of the needs presented above, the present invention in one embodiment relates a personal care kit comprising a first series of unit-dose packages containing a first skin care composition and a second series of unit-doses packages containing a second skin care composition, wherein the first skin care composition and the second skin care composition exhibit a readily perceptible difference. The readily perceptible difference may be a visible difference, a tactile difference, or an olfactory difference. The first and/or second skin care composition may comprise a skin care active.

By way of further addressing the needs presented above, the present invention in one embodiment relates to a method for treating the skin comprising the steps of (a) dispensing and applying a first skin care composition from a first unit-dose package; (b) repeating step (a) for a plurality of times at a first interval; (c) dispensing and applying a second skin care composition from a second unit-dose package; (d) repeating step (c) at a second routine interval; wherein step (c) begins after the plurality of times. The application of the skin care composition may occur daily for a number of days such as for 7 days.

### BRIEF DESCRIPTION OF THE DRAWINGS

FIGS. 1A-F are suitable embodiments of unit-dose packages.

FIG. 2 depicts a suitable personal care kit.

FIG. 3A is a top view of a suitable personal care kit.

FIG. 3B is a cross-sectional view taken through line A-A of FIG. 3A.

### DETAILED DESCRIPTION OF THE INVENTION

As used herein, the following terms shall have the meaning specified thereafter. It should be recognized that capitalization of the listed terms is predicated on grammar and may not be continued throughout the remaining of the text.

“Series” refers to a plurality of packages (e.g., containers, vials, vessels, cans, cartons, canisters, tubes, or like devices) containing the same skin care composition wherein the plurality are used in succession and without intervening use of another, different skin care composition, wherein both skin care compositions are provided within a personal care kit.

“Applied” or “application”, unless explicitly stated otherwise, means to spread a composition onto keratinous tissue with one or more fingers and/or an implement, as one would be expected to apply a cream to the facial skin.

“Perceptible difference” refers to a difference between one skin care composition and another skin care composition that is perceptible to a person having normal sensory abilities (e.g., 20/20 vision, normal hearing, unimpaired tactile sensation, etc.) under standard conditions. For example, standard lighting conditions are at least one of the following: natural illumination as experienced outdoors during daylight hours, the illumination of a standard 100 watt incandescent white light bulb at a distance of 2 meters, or as defined by CIE D65 standard illuminate lighting at 800 lux to a 1964 CIE standard observer.

“Readily perceptible difference” refers to a perceptible difference that is perceptible before, during, or immediately after (i.e., less than 5 minutes) application of the skin care compositions.

“Post-application skin temperature change” refers to the absolute value of the difference between the skin temperature measured immediately before application of a skin care composition and the skin temperature measured 1 minute after application of the skin care composition. Prior to application, the skin care composition is equilibrated to the pre-application skin temperature.

“Personal care composition” means a composition suitable for topical application on mammalian keratinous tissue.

“Dose” refers the amount of skin care composition to be used during application.

“Unit-dose package” refers to a package (e.g., container, vial, vessel, ampule, pouch, can, carton, canister, capsule, tube, substrate, wipe, pad, cup, blister pack, or like devices) containing a single dose of a skin care composition which is to be completely dispensed and applied.

“Bulk package” refers to a package containing a plurality of doses of a skin care composition.

“Skin care actives,” or “actives,” as used herein, means compounds that, when applied to the skin, provide a benefit or improvement to the skin. Skin care actives may be used for regulating skin condition, improving skin condition, It is to be understood that skin care actives are useful not only for application to skin, but also to hair, nails, and other mammalian keratinous tissue.

“Keratinous tissue” refers to keratin-containing layers disposed as the outermost protective covering of mammals which includes, but is not limited to, skin, hair, nails, cuticles, etc.

“Regulating skin condition” means maintaining skin appearance and/or feel with little to no degradation in appearance and/or feel. “Improving skin condition” means affecting a positive change in skin appearance and/or feel. The skin appearance and/or feel benefit may be a chronic benefit and may include one or more of the following: thickening of keratinous tissue (e.g., building the epidermis and/or dermis and/or sub-dermal layers of the skin, and where applicable the keratinous layers of the nail and hair shaft, to reduce skin, hair, or nail atrophy); increasing the convolution of the dermal-epidermal border (also known as the rete ridges); preventing loss of skin or hair elasticity, for example, due to loss, damage and/or inactivation of functional skin elastin, resulting in such conditions as elastosis, sagging, loss of skin or hair recoil from deformation; reduction in cellulite; change in coloration to the skin, hair, or nails, for example, under-eye circles, blotchiness (e.g., uneven red coloration due to, for example, rosacea), sallowness, discoloration caused by hyperpigmentation, maintaining/improving the signs of skin aging, and maintaining/improving insult-affected keratinous tissue.

“Signs of skin aging,” include, but are not limited to, all outward visibly and tactilely perceptible manifestations, as well as any macro- or micro-effects, due to keratinous tissue aging. These signs may result from processes which include, but are not limited to, the development of textural discontinuities such as wrinkles and coarse deep wrinkles, fine lines, skin lines, crevices, bumps, large pores, unevenness or roughness; loss of skin elasticity; discoloration (including undereye circles); blotchiness; sallowness; hyperpigmented skin regions such as age spots and freckles; keratoses; abnormal differentiation; hyperkeratinization; elastosis; collagen breakdown, and other histological changes in the stratum corneum, dermis, epidermis, vascular system (e.g., telangiectasia or spider vessels), and underlying tissues (e.g., fat and/or muscle), especially those proximate to the skin.

“Insult-affected keratinous tissue,” means keratinous tissue which exhibits discomfort, irritation, an unpleasant or irregular appearance, and the like, for example after exposure to a physical and/or chemical irritant. Non-limiting examples of insult-affected keratinous tissue include burn (e.g., sunburns, windburn, chemical or thermal burns); rashes (e.g., diaper rash, shaving rash and allergen-induced rashes); discoloration (e.g., bleaching, staining, hyperpigmentation); nicks and cuts (e.g., shaving insults); and dry, chapped or rough skin (e.g., due to exposure to example wind, cold and/or low humidity). Non-limiting examples of insults include radiation, wind, low humidity, allergens, pollutants, chemical and natural irritants, bodily fluids, bodily waste, excessive moisture, bacteria, fungi, etc.

“Safe and effective amount” means an amount of a compound or composition sufficient to induce a positive benefit but low enough to avoid serious side effects (i.e., provides a reasonable benefit to risk ratio within the judgment of a skilled artisan).

“Substantially free,” when used in reference to a substance that facilitates a readily perceptible difference, refers to an amount of the substance within a skin care composition that is undetectable to a person having normal sensory abilities. Substantially free by definition includes 0% of a substance.

In all embodiments of the present invention, all percentages are by weight of the total composition, unless specifically stated otherwise. All ratios are weight ratios, unless specifically stated otherwise. All ranges are inclusive and combinable; therefore, every range given throughout this specification will include every narrower range that falls within such broader range as if such narrower ranges were all expressly written herein. The number of significant digits conveys neither a limitation on the indicated amounts nor on the accuracy of the measurements. Unless explicitly stated otherwise, all measurements are understood to be made at 25° C. and at ambient conditions, where “ambient conditions” means conditions under about one atmosphere of pressure and at about 50% relative humidity. All such weights as they pertain to listed ingredients are based on the active level and do not include carriers or by-products that may be included in commercially available materials, unless otherwise specified.

### I. Personal Care Kit

The personal care kit of the present invention may be used for the care of keratinous tissue. The personal care kit of the present invention may be used for the care of skin of the whole body or the face. The personal care kit of the present invention may be used for care of discrete spots of skin. The personal care kit may regulate and/or improve skin condition. The personal care kit may regulate and/or improve signs of skin aging or insult-affect keratinous tissue.

The personal care kit comprises a first series of unit-dose packages containing a first skin care composition and a second series of unit-dose packages containing a second skin care composition. All of the first series is applied and used prior to application and use of the second series; conversely, in other embodiments, all of the second series may be applied and used first prior to the application and use of the first series. It is further contemplated that the first and second skin care compositions may be used in parallel (i.e., a repeating application regime of the first skin care composition and the second skin care composition). The first skin care composition and the second skin care composition exhibit a readily perceptible difference.

The particular number of unit-dose packages comprising the first skin care composition or the second skin care composition is not limited. In certain embodiments, the kit comprises a plurality of unit-dose packages comprising the first skin care composition and a plurality of unit-dose packages comprising the second skin care composition. In other embodiments, the kit may comprise 7, 14, or 21 unit-dose packages comprising the first skin care composition and 7, 14, or 21 unit-dose packages comprising the second skin care composition.

The dosing interval of the first skin care composition and the second skin care composition is not limited. The dosing interval may be selected based on the composition of the skin care composition and, if present, skin care active within in the composition. Customary dosing intervals for topical application of skin care composition include once daily (e.g., applied once each day at approximately the same time) and twice daily (e.g., applied twice each day at approximately the same time and at approximately equal intervals). However, other dosing intervals are contemplated (e.g., frequency of more or less than once per week). In one embodiments, a first skin care composition from a first unit-dose package is applied once a day for m number of says and then, after all of the first unit-dose packages have been used, a second skin care composition from a second unit-dose package is applied once a day for n number of days.

The kit is not limited in construction or physical form. In certain embodiments, the kit may be in the form of a consumer unit. A “consumer unit” is a single entity for consumer sale that contains the components of the kit. The consumer unit may comprise several packages, boxes, or other like containers that are joined to form a single entity (e.g., several smaller packages contained within a larger container, several packages bound or adhered to for a single entity, etc.). For example, the first series of unit-dose packages containing a first skin care composition may be provided in a first container, the second series of unit-dose packages containing a second skin care composition may be provided in a second container, and the first container and second container may be joined to form the consumer unit.

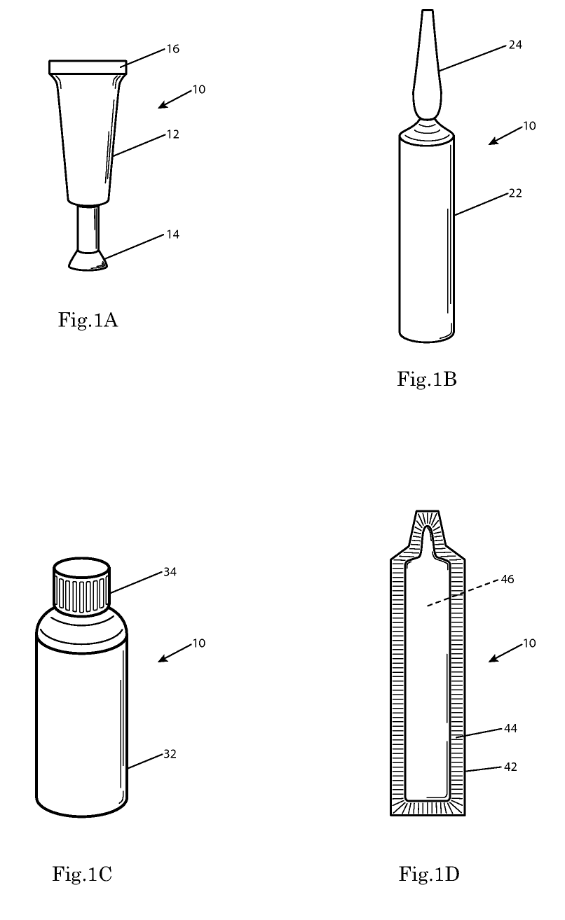

A. Unit-Dose Package—As defined above, a unit-dose package refers to a package containing a single dose of a skin care composition which is to be completely dispensed and applied. The particular form of the unit-dose package may be of any suitable form such as, but not limited to, a container, vial, vessel, ampule, pouch, sachet, can, carton, canister, capsule, tube, substrate, wipe, pad, cup, blister pack, or like devices. In certain embodiments, the unit-dose package may be gas-impermeable and/or liquid-impermeable. In certain embodiments, the unit-dose package is constructed to prevent inadvertent dispensation of the skin care composition. FIG. 1A depicts one particular embodiment of a unit-dose package (10) in the form of a tube (12) that has a frangible seal (14) on one end and a crimped seal (16) on the opposing end. In the aforementioned design, the frangible seal (14) is twisted-off thereby enabling a user to dispense the composition by squeezing on the tube. FIG. 1B depicts a unit-dose package (10) in the form of an ampule (22) having a frangible tip (24). FIG. 1C is another embodiment of a unit-dose package (10) in the form of a vial (32) with a removable lid (34). FIG. 1D is another embodiment of a unit-dose package (10) in the form of a sachet (42) formed from two substrates that are perimeter-sealed (44) to form an inner void (46). FIG. 1E is another embodiments of a unit-dose package (10) in the form of a moisten wipe (52). The wipe (52) is shown as being contained within a liquid impermeable pouch (54) with a portion of the pouch removed to show the underlying wipe (52). In other embodiments, the wipe (52) may be a dry wipe. Furthermore, the wipe (52) may be container in various other overwraps or containers. For example, a plurality of wipes (52) may be stored in a jar or like container. Other suitable substrate-based unit-dose packages are shown in U.S. Pat. No. 5,616,337 and in U.S. Publication 2004/0109720. FIG. 1F depicts another embodiment of unit-dose package (10) in the form of a dispensing applicator (62). The dispensing applicator (62) comprises two substrates that are perimeter-sealed (64) to form a reservoir (66). The reservoir (66) opens to a substrate (68) joined to one end of the dispensing applicator (62). The substrate (68) is placed in contact with the skin to deliver the skin care composition. The substrate (68) may be any suitable skin-friendly material such a nonwoven web, a woven fabric, a foam, and the like. U.S. Pat. No. 6,902,335 provides additional discussion of the dispensing applicator (62).

In another embodiment, the unit-dose package may be in the form of a swab-type applicator such as disclosed in U.S. Pat. Nos. 3,958,571, 4,173,978, and 4,799,815. Other suitable unit-dose packages are described in U.S. Pat. Nos. 4,257,838, 4,342,395, 4,871,091, 4,951,822, 4,979,630, 5,063,057, 5,270,054, 5,503,885, and 5,535,885 and in U.S. Design Pat. Nos. 515,936, 505,861, 488,375, 467,336, 463,275, 446,445, 443,505, 442,078, 435,440, 425,617, 411,296, 392,184, 369,212, 362,390, 347,995, 331,009, 327,008, 324,333, 318,795, 296,869, and 288,061.

The prescribed amount of skin care composition provided in the unit-dose package is not limited. The dose of skin care composition contained in unit-dose package may depend on the target area. For example, in certain embodiments, the unit-dose package may be constructed to deliver a dose of about 30-25 mL for full body application, about 5-1 mL for facial application, and less than about 1 mL for spot treatment. However, a smaller or larger dose may be delivered depending on the skin condition and components of the skin care composition.

In one embodiment, there is a difference in the packaging between said products. These differences may be in aesthetic for example the color, shape or appearance of the packaging. For example, in the tubes of the present invention it would be possible to have a different heat seal in the end of the tube such as a straight heat seal and a curved heat seal. The printing could also be matte versus glossy. Furthermore the packaging could include a number or other visual indicators that they are different including graphics, printing, etc.

Within a series of unit-dose packages, the individual unit-dose packages may be similar or different. Likewise, between two or more series of unit-dose packages, the individual unit-dose packages within the first series may be similar or different from the individual unit-dose packages within the second series. In an exemplary embodiment, the unit-dose packages of the first series may be in the form of a container having an applicator such as depicted in the aforementioned U.S. Pat. No. 6,902,335 and the unit-dose packages of the second series may be in the form of a tube such as depicted in the aforementioned U.S. Design Pat. No. 288,061. The unit-dose packages within the skin care kit of the present invention may supplied separately or may be interconnected. The unit-dose packages may be interconnected directly (i.e., one unit-dose package is directly joined to another unit-dose package) or indirectly (e.g., one unit-dose package is joined to a substrate or housing that also joined one or more other unit-dose packages).

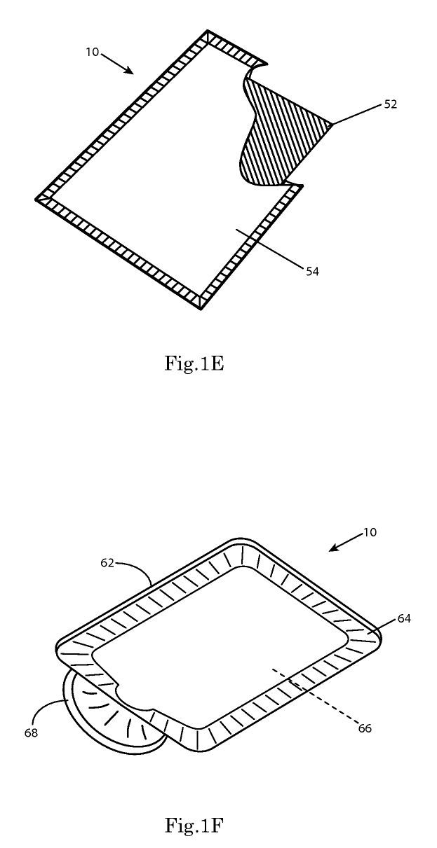

In one embodiment of the personal care kit (200), as shown in FIG. 2, the unit-dose packages (210) of the first series and the second unit-dose packages (220) of the second series are housed in a tray (230) have suitable recesses (232) to accept the unit-dose packages (210, 220). The unit-dose packages (210) of the first series and the second unit-dose packages (220) of the second series may be arranged to facilitate compliance with the successive application of the series.

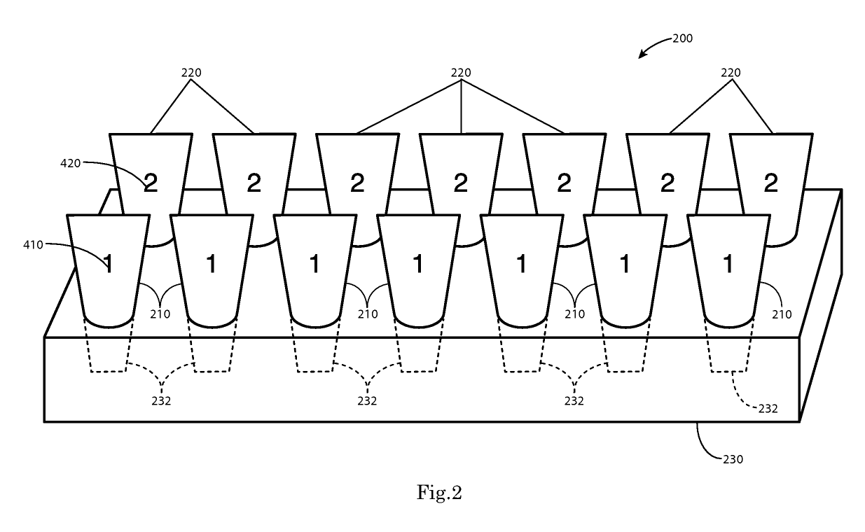

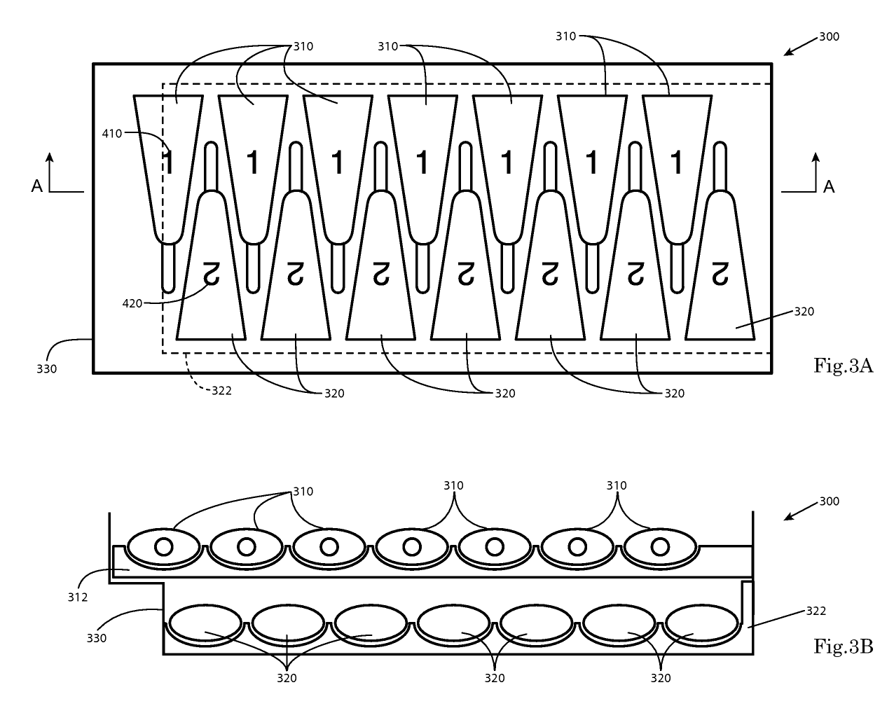

In another embodiment the personal care kit (300), as shown in FIGS. 3A-B, the unit-dose packages (310) of the first series are joined to a first housing (312). The unit-dose packages (320) of the second series are joined to a second housing (322). The first housing (312) and second housing (322) may secure their respective unit-dose packages (310, 320). The first housing (312) and second housing (322) may be constructed to provide a recess into which the respective unit-dose packages (310, 320) fit. The first housing (312) and second housing (322) may prevent repositioning of the respective unit-dose packages (310, 320) during normal shipping and handling of the personal care kit (300). FIG. 3A shows a top-view of the personal care kit (300). FIG. 3B shows a cross-sectional view taken through sectional line A-A of FIG. 3A. FIG. 3B shows a suitable arrangement of the unit-dose packages (310) of the first series joined to the first housing (312) and unit-dose packages (320) of the second series joined to the second housing (322). The first and second housings (312, 322) may be disposed within tray (330). The tray (330) may assist in maintaining the relative position of the unit-dose packages (310, 320). The first and second housings (312, 322) may be disposed such that the first housing (312) is more readily accessible than the second housing (322) to a user. As shown in FIG. 3B, the first housing (312) may be positioned on top of the second housing (322). Such a positioning facilities the successive use of the series. A user will most likely use the unit-dose packages (310) of the first series and then remove the first housing (312) thereby gaining access to the unit-dose packages (320) of the second series. The first and second housing (312, 322) may be discrete or integral.

In certain embodiments, the unit-dose packages of the first series have a first indicium (410) and the unit-dose packages of the second series have a second indicium (420) different from the first indicium (as shown in FIGS. 2 and 3A-B). “Indicium” refers to any type of visible identifying mark or property of the unit-dose package. Suitable indicia include the shape, color, design, print, graphics, text, numbers, and the like. In one embodiment, the first series of unit-dose packages have a color different from the color of the unit-dose packages in the second series. In another embodiment, the first series of unit-dose packages may be printed with a first use indicium and the second series of unit-dose packages may be printed with a second use indicium. The first use indicium and second use indicium are indicia that convey a sequence such that the first use indicium precedes the second use indicium within the sequence. Suitable first use and second use indicium include numbers, and letters. In one embodiment as shown in FIGS. 2 and 3A-B, the first series of unit-dose packages may be printed with a first use indicium (410) of the number “1” and the second series of unit-dose packages may be printed with a second use indicium (420) of the number “2”.

B. Skin Care Compositions—The skin care compositions of the present invention may comprise a dermatologically acceptable carrier and, optionally, one or more skin care actives.

(i) Dermatologically Acceptable Carrier—The skin care compositions of the present invention can also comprise a dermatologically acceptable carrier (“carrier”) for the composition. In one embodiment, the carrier is present at a level of from about 50% to about 100%, about 60% to about 99.9%, about 70% to about 98%, or, alternatively, from about 80% to about 95%, by weight of the composition.

The carrier can be in a wide variety of forms. Non-limiting examples include simple solutions (water or oil based), emulsions, and solid forms (gels, sticks, flowable solids, amorphous materials). In certain embodiments, the dermatologically acceptable carrier is in the form of an emulsion. Emulsion may be generally classified as having a continuous aqueous phase (e.g., oil-in-water and water-in-oil-in-water) or a continuous oil phase (e.g., water-in-oil and oil-in-water-in-oil). The oil phase of the present invention may comprise silicone oils, non-silicone oils such as hydrocarbon oils, esters, ethers, and the like, and mixtures thereof. For example, emulsion carriers can include, but are not limited to, continuous water phase emulsions such as silicone-in-water, oil-in-water, and water-in-oil-in-water emulsion; and continuous oil phase emulsions such as water-in-oil and water-in-silicone emulsions, and oil-in-water-in-silicone emulsions.

The aqueous phase typically comprises water. However, in other embodiments, the aqueous phase may comprise components other than water (non-water components), including but not limited to water-soluble moisturizing agents, conditioning agents, anti-microbials, humectants and/or other water-soluble skin care actives, to impart an increased benefit to the keratinous tissue. In one embodiment, the non-water component of the composition comprises a humectant such as glycerin and/or other polyols. However, it should be recognized that the carrier may be substantially (i.e., less than 1% water) or fully anhydrous.

A skilled artisan will select a suitable carrier to yield a desired product form. Furthermore, a skilled artisan will select a suitable carrier to for any given skin care active or other component will distribute primarily into either the water or oil phase, depending on the water solubility/dispensability of the component in the composition. In one embodiment, oil-in-water emulsions are especially preferred.

Emulsions may further comprise an emulsifier. The skin care composition may comprise from about 0.1% to about 10% or about 0.2% to about 5% of an emulsifier, based on the weight of the composition. Emulsifiers may be nonionic, anionic or cationic. Suitable emulsifiers are disclosed in, for example, U.S. Pat. No. 3,755,560, U.S. Pat. No. 4,421,769, and McCutcheon's Detergents and Emulsifiers, North American Edition, pages 317-324 (1986). Suitable emulsions may have a wide range of viscosities, depending on the desired product form.

The carrier may further comprise a thickening agent as are well known in the art to provide a skin care compositions yielding a suitable viscosity and rheological character.

(ii) Skin Care Active—The skin care composition of the present invention may comprise a safe and effective amount of one or more skin care active (“active”) useful for regulating and/or improving skin condition. Suitable actives include, but are not limited to, vitamins, peptides, sugar amines, sunscreens, oil control agents, tanning actives, anti-acne actives, desquamation actives, anti-cellulite actives, chelating agents, skin lightening agents, flavonoids, protease inhibitors, non-vitamin antioxidants and radical scavengers, hair growth regulators, anti-wrinkle actives, anti-atrophy actives, minerals, phytosterols and/or plant hormones, tyrosinase inhibitors, anti-inflammatory agents, N-acyl amino acid compounds, antimicrobials, and antifungals. The skin care composition may comprise of the

“Vitamins” means vitamins, pro-vitamins, and their salts, isomers and derivatives. Non-limiting examples of suitable vitamins include: vitamin B compounds (including B1 compounds, B2 compounds, B3 compounds such as niacinamide, niacinnicotinic acid, tocopheryl nicotinate, C1-C18 nicotinic acid esters, and nicotinyl alcohol; B5 compounds, such as panthenol or “pro-B5”, pantothenic acid, pantothenyl; B6 compounds, such as pyroxidine, pyridoxal, pyridoxamine; carnitine, thiamine, riboflavin); vitamin A compounds, and all natural and/or synthetic analogs of Vitamin A, including retinoids, retinol, retinyl acetate, retinyl palmitate, retinoic acid, retinaldehyde, retinyl propionate, carotenoids (pro-vitamin A), and other compounds which possess the biological activity of Vitamin A; vitamin D compounds; vitamin K compounds; vitamin E compounds, or tocopherol, including tocopherol sorbate, tocopherol acetate, other esters of tocopherol and tocopheryl compounds; vitamin C compounds, including ascorbate, ascorbyl esters of fatty acids, and ascorbic acid derivatives, for example, ascorbyl phosphates such as magnesium ascorbyl phosphate and sodium ascorbyl phosphate, ascorbyl glucoside, and ascorbyl sorbate; and vitamin F compounds, such as saturated and/or unsaturated fatty acids.

The compositions of the present invention may comprise one or more peptides. Herein, “peptide” refers to peptides containing ten or fewer amino acids, their derivatives, isomers, and complexes with other species such as metal ions (for example, copper, zinc, manganese, and magnesium). As used herein, peptide refers to both naturally occurring and synthesized peptides. In one embodiment, the peptides are di-, tri-, tetra-, penta-, and hexa-peptides, their salts, isomers, derivatives, and mixtures thereof. Suitable peptides include, but are not limited to, peptides derived from soy proteins, carnosine, palmitoyl-lysine-threonine (pal-KT) and palmitoyl-lysine-threonine-threonine-lysine-serine (pal-KTTKS, available in a composition known as MATRIXYL®), palmitoyl-glycine-glutamine-proline-arginine (pal-GQPR, available in a composition known as RIGIN®), these three being available from Sederma, France, acetyl-glutamate-glutamate-methionine-glutamine-arginine-arginine (Ac-EEMQRR; Argireline®), and Cu-histidine-glycine-glycine (Cu-HGG, also known as IAMIN®).

The compositions of the present invention may comprise a sugar amine, also known as amino sugars, and their salts, isomers, tautomers and derivatives. Sugar amines can be synthetic or natural in origin and can be used as pure compounds or as mixtures of compounds (e.g., extracts from natural sources or mixtures of synthetic materials). Sugar amine compounds useful in the present invention include, for example, N-acetyl-glucosamine, and also those described in PCT Publication WO 02/076423 and U.S. Pat. No. 6,159,485.

The compositions of the subject invention may comprise one or more sunscreen actives (or sunscreen agents). Herein, suitable sunscreen actives include oil-soluble sunscreens, insoluble sunscreens, and water-soluble sunscreens. Non-limiting examples of suitable oil-soluble sunscreens are disclosed in The Cosmetic, Toiletry, and Fragrance Association's The International Cosmetic Ingredient Dictionary and Handbook, 10^{th} Ed., Gottschalck, T. E. and McEwen, Jr., Eds. (2004), p. 2267 and pp. 2292-93 and include benzophenone-3, bis-ethylhexyloxyphenol methoxyphenyl triazine, butyl methoxydibenzoyl-methane, diethylamino hydroxy-benzoyl hexyl benzoate, drometrizole trisiloxane, ethylhexyl methoxy-cinnamate, ethylhexyl salicylate, ethylhexyl triazone, octocrylene, homosalate, polysilicone-15, and derivatives and mixtures thereof. Non-limiting examples of suitable insoluble sunscreens include methylene bis-benzotriazolyl tetramethylbutyl-phenol, titanium dioxide, zinc cerium oxide, zinc oxide, and derivatives and mixtures thereof. Non-limiting examples of suitable water-soluble sunscreens include phenylbenzimidazole sulfonic acid (PBSA), terephthalylidene dicamphor sulfonic acid, (Mexoryl™ SX), benzophenone-4, benzophenone-5, benzylidene camphor sulfonic acid, cinnamidopropyl-trimonium chloride, methoxycinnamido-propyl ethyldimonium chloride ether, disodium bisethylphenyl triaminotriazine stilbenedisulfonate, disodium distyrylbiphenyl disulfonate, disodium phenyl dibenzimidazole tetrasulfonate, methoxycinnamido-propyl hydroxysultaine, methoxycinnamido-propyl laurdimonium tosylate, PEG-25 PABA (p-aminobenzoic acid), polyquaternium-59, TEA-salicylate, and salts, derivatives and mixtures thereof.

The compositions of the present invention may comprise one or more compounds for regulating the production of skin oil, or sebum, and for improving the appearance of oily skin. Examples of suitable oil control agents include salicylic acid, dehydroacetic acid, benzoyl peroxide, vitamin B3 compounds (for example, niacinamide or tocopheryl nicotinate), their isomers, esters, salts and derivatives, and mixtures thereof.

The compositions of the present invention may comprise a tanning active such as dihydroxyacetone, which is also known as DHA or 1,3-dihydroxy-2-propanone.

The compositions of the present invention may comprise a safe and effective amount of one or more anti-acne actives. Examples of useful anti-acne actives include resorcinol, sulfur, salicylic acid, erythromycin, zinc, etc. Suitable anti-acne actives are described in further detail in U.S. Pat. No. 5,607,980.

The compositions of the present invention may comprise a safe and effective amount of a desquamation system such as sulfhydryl compounds and zwitterionic surfactants as described in U.S. Pat. No. 5,681,852. Another suitable desquamation system comprises salicylic acid and zwitterionic surfactants and is described in U.S. Pat. No. 5,652,228.

The compositions of the present invention may comprise a safe and effective amount of an anti-cellulite agent including, but are not limited to, xanthine compounds (e.g., caffeine, theophylline, theobromine, and aminophylline).

The compositions of the present invention may comprise a safe and effective amount of a chelating agent as disclosed in U.S. Pat. No. 5,487,884. A suitable chelator is furildioxime and derivatives.

The compositions of the present invention may comprise a skin lightening agent including kojic acid, arbutin, tranexamic acid, ascorbic acid and derivatives (e.g., magnesium ascorbyl phosphate or sodium ascorbyl phosphate or other salts of ascorbyl phosphate), ascorbyl glucoside, undecylenoyl phenylalanine (Sepiwhite® from SEPPIC), aloesin, Actiwhite® (Cognis), and Emblica® (Rona).

The compositions of the present invention may comprise a flavonoid. The flavonoid can be synthetic materials or obtained as extracts from natural sources, which also further may be derivatized. Examples of classes of suitable flavonoids are disclosed in U.S. Pat. No. 6,235,773.

The compositions of the present invention may comprise protease inhibitors including, but are not limited to, hexamidine (including salts and derivates thereof), vanillin acetate, menthyl anthranilate, soybean trypsin inhibitor, Bowman-Birk inhibitor, and mixtures thereof.

The compositions of the present invention may other optional components such as non-vitamin antioxidants and radical scavengers, hair growth regulators, anti-wrinkle actives, anti-atrophy actives, minerals, phytosterols and/or plant hormones, tyrosinase inhibitors, anti-inflammatory agents, N-acyl amino acid compounds, antimicrobial or antifungal actives, and other useful skin care actives, which are described in further detail in U.S. application publication No. US2006/0275237A1 and US2004/0175347A1.

C. Readily Perceptible Difference—The personal care kit of the present invention comprises a first series of unit-dose packages containing a first skin care composition and a second series of unit-dose packages containing a second skin care composition wherein the first skin care composition and the second skin care composition exhibit a readily perceptible difference. A readily perceptible difference may be any difference between the first skin care composition and the second skin care composition that is perceptible to a person having normal sensory abilities before, during, or immediately after (i.e., less than 5 minutes) application of the skin care compositions. The readily perceptible difference may be appreciable before application such as by visual inspection of the skin care compositions with the unit-dose packages. The readily perceptible difference may be appreciable during application such as when the skin care composition is dispensed on a user's fingers or area of skin targeted for application. The readily perceptible difference may be appreciable immediately after application of the skin care composition to the area of skin targeted for application. In certain embodiments, the readily perceptible difference is a tactile difference, a visible difference, an olfactory difference, or any combination thereof. The following readily perceptible differences represent suitable embodiments of the present invention; however, the present invention further contemplates any combination of two or more of the following readily perceptible differences.

(i) Tactile Differences—The personal care kit of the present invention comprises a first series of unit-dose packages containing a first skin care composition and a second series of unit-dose packages containing a second skin care composition wherein the first skin care composition and the second skin care composition exhibit a readily perceptible tactile difference. The tactile difference may be any difference between the first skin care composition and the second skin care composition that is appreciable by the sense of touch (including pressure, hardness, temperature, smoothness, greasiness, etc.). Exemplary tactile differences are provided below.

(a) Viscosity—The first skin care composition may have a higher or lower viscosity than the second skin care composition. The resulting tactile difference is of one composition feeling thin and the other composition feeling thick. In certain embodiments, the two skin care compositions differ by at least about 5,000 centipoise (cP), 10,000 cP, or, alternatively, 20,000 cP. In one embodiment, the first skin care composition exhibits a viscosity of less than 40,000 cP and the second skin care composition exhibits a viscosity of more than 40,000 cP. In another embodiment, the first skin care composition exhibits a viscosity of less than 30,000 cP and the second skin care composition exhibits a viscosity of more than 50,000 cP.

(b) Skin Temperature—The first and second skin care compositions may exhibit a tactile difference via a post-application skin temperature change. In one embodiment, the first skin care composition results in a post-application skin temperature change of at least about 1° C., about 2° C., or, alternatively, about 5° C.; whereas the second skin care composition results in a post-application skin temperature change of less than about 0.25° C. or about 0.10° C. or no change.

In other embodiments, the first skin care composition may exhibit a post-application skin temperature increase of at least about 1° C., about 2° C., or, alternatively, about 5° C. and the second skin care composition may exhibit a post-application skin temperature decrease of at least about 1° C., about 2° C., or, alternatively, about 5° C.

Skin temperatures are measured using a high precision hand-held infrared thermographic scanner such as the DermaTemp DT-1001 available from Exergen Corp., Watertown, Mass. The skin temperature measurements may be performed on any two symmetrically opposing (i.e., sinister and dexter) skin surfaces (“sample site”). For example, the skin temperatures may be measured on the left and right temple, cheek, forearm, shoulder, etc. A 1 inch by 1 inch sample site is delineated (e.g., indelible ink or medical tape) on the left and right skin surface. A pre-application skin temperature is recorded for the left and right sample sites approximately 5 minutes prior to application of the skin care compositions. A dose of the first skin care composition is applied to one sample site and the second skin care composition is applied to the other sample site. The skin care compositions are equilibrated to a temperature of 75° F. prior to application. One minute after application, a post-application skin temperature is recorded for the left and right sample sites. The post-application skin temperature change is calculated by taking the absolute value of the difference of the post-application skin temperature and the pre-application skin temperature (i.e., |post−pre|). However, a post-application skin temperature increase and post-application skin temperature decrease are calculated by as the difference between the post-application skin temperature and the pre-application skin temperature (i.e., post−pre). The skin care compositions may comprise about 5% to about 60% of metal chlorides (e.g., calcium chloride, magnesium chloride, aluminum chloride, ferric chloride), metal sulfates (e.g., calcium sulfate, magnesium sulfate, aluminum sulfate, ferric sulfate), dry alum, calcium oxide, and other as described in U.S. Publication 2004/0022823, which may provide a post-application temperature change.

(c) Sensates—The skin care compositions may exhibit a tactile difference through the use of warming sensates or cooling senates. Sensates provide the sensation of heating or cooling to a user, but may or may not yield a change in skin temperature. The sensation may be instantaneous or may be delayed, but, generally, is appreciable within 5 minutes of application of the skin care composition.

Suitable warming sensates include vanillyl alcohol derivatives including of vanillyl alcohol n-butyl ether, vanillyl alcohol npropyl ether, vanillyl alcohol isopropyl ether, vanillyl alcohol isobutyl ether, vanillyl alcohol n-amino ether, vanillyl alcohol isoamyl ether, vanillyl alcohol n-hexyl ether, vanillyl alcohol methyl ether and vanillyl alcohol ethyl ether; and phosphate derivatives comprising the aforementioned vanillyl alcohol derivatives such as vanillyl alcohol isoamyl ether monophosphate, vanillyl alcohol n-butyl ether monophosphate, vanillyl alcohol n-hexyl ether monophosphate. Other suitable warming sensates include ethyl alcohol, niacin, jambu, nicotinic acid, zingerone, vanillyl alcohol n-butyl ether, vanillyl alcohol n-propyl ether, vanillyl alcohol isopropyl ether, vanillyl alcohol isobutyl ether, vanillyl alcohol n-amino ether, vanillyl alcohol isoamyl ether, vanillyl alcohol n-hexyl ether, vanillyl alcohol methyl ether, vanillyl alcohol ethyl ether, gingerol, methyl salicylate, shogaol paradol, zingerone, capsaicin, dihydrocapsaicin, nordihydrocapsaicin, homocapsaicin, homodihydrocapsaicin, ethanol, tincture capsicum, oleoresin ginger alcohol extraction, eucalyptus oil, capsaicin, cinnamic aldehyde, chloroform, ether, iso-Amyl alcohol, benzyl alcohol, allyl isothiocyanate, ethyl acetate, glycerine, limonene, menthol, 4-hydroxy-4-methyl-cyclohexen-2-one-1, and mixtures thereof. Further suitable warming sensates include fluid extracts, hydro-alcohol extracts, essential oils, oleoresins, concretes or distillates of mustard seed, ginger, horseradish, chillies, jalapeno, pepper, capsicum, clove, cassia, and mixtures thereof. Any mixture of the aforementioned warming sensates may also be used.

Suitable cooling sensates include menthol, isopulegole, 3-(1-menthoxy)propan-1,2-diol, p-menthan-3,8-diol, 6-isopropyl-9-methyl-1,4-dioxaspiro-(4,5)-decane-2-methanol, menthyl succinate, alkaline earth salts of menthyl succinate, trimethyl cyclohexanol, N-ethyl-2-isopropyl-5-methylcyclohexane carboxamide and other carboxamides as described in U.S. Pat. Nos. 4,136,163 and 4,230,688, 3-(1-menthoxy)-2-methyl-propan-1,2-diol, mint oil, peppermint oil, wintergreen, menthone, menthone glycerin ketal and other glycerol ketals described in U.S. Pat. No. 5,266,592, menthyl lactate, 2-(5′-methyl-2-(methylethyl)cyclohexyloxy)ethan-1-ol, 3-(5′-methyl-2′-(methylethyl)cyclohexyloxy)propan-1-ol, 4-(5′-methyl-2′-(methyl ethyl)cyclohexyloxy)butan-1-ol, and spearmint. Other cooling sensates include p-menth-3-yl n-butyl sulphoxide, n-butyl 1-isobutylcyclohexyl sulphoxide, n-hexyl 1-isobutylcyclohexyl sulphoxide, n-butyl 1-isoamylcyclohexyl sulphoxide and n-hexyl 1,2-diethylcyclohexyl sulphoxide, and other cyclic sulphoxides and sulphones as described in U.S. Pat. No. 4,032,661.

The skin care compositions may comprise from about 0.001% to about 25% of one or more warming sensates or cooling sensates. In one embodiment of the present invention, the first skin care composition may comprise one or more warming sensates or one or more cooling sensates and the second skin care composition is substantially free of a warming sensate or cooling sensate. In another embodiment of the present invention, the first skin care composition may comprise one or more warming sensates and the second skin care composition comprises one or more cooling sensates.

(d) Abrasives—The skin care compositions may exhibit a tactile difference through the use of particulate material having a prescribed average particle size. The resulting tactile difference is of one composition feeling abrasive or gritty and the other composition feeling smooth. In a suitable embodiment, the first skin care composition comprises an abrasive particulate material having an average particle size of greater than about 100 μm and the second skin care composition is substantially free of an abrasive particulate material having an average particle size of greater than about 100 μm. In other embodiments, the first skin care composition comprises an abrasive particulate material having an average particle size of greater than about 150 μm, 200 μm, or 250 μm and the second skin care composition is substantially free of an abrasive particulate material having an average particle size of greater than about 100 μm. In certain embodiments, the skin care composition may comprise from about 0.1% to about 20% of the abrasive particulate material that provides the abrasive sensation.

Particulate materials useful herein include but are not limited to bismuth oxychloride, sericite, mica, mica treated with barium sulfate or other materials, zeolite, kaolin, silica, boron nitride, lauroyl lysine, polyamide (e.g., nylon-12 and Orgasol™ 2002D Nat C05 available from Atofina), polyolefins including polyethylene (e.g., Microthene™ line available from Equistar and PFL (500-850 μm), PFM (250-500 μm), and PFS (100-250 μm) colored scrubbing beads from Kobo) and polypropylene, talc, styrene, polystyrene (e.g., Dynospheres™ available from Dyno Particles), ethylene/acrylic acid copolymer, sericite, aluminum oxide, silicone resin, barium sulfate, calcium carbonate, cellulose acetate, polytetrafluoroethylene (PTFE), polyacrylates (e.g., ethylene acrylate copolymer, sold under the name FloBead™ EA209 available from Kobo), polymethyl methacrylate (e.g., Micropearl™ M 100 available from Seppic), starch, modified starches such as aluminum starch octenyl succinate, silk, glass, fibers, ground seeds, pumice, and mixtures thereof.

(e) Oil Content—The first skin care composition may comprise a higher or lower oil content than that of the second skin care composition. The resulting tactile difference is of one composition feeling oily or greasy compared to the other composition. In one embodiment, the first skin care composition has an oil content of at least 30% and the second skin care content has an oil content of less than 30%. In other embodiments, the first skin care composition may have an oil content of at least 40%, 50%, or 60% and the second skin care content has an oil content of less than 25%, 20%, or 15%. The oil content is weight percentage, based on the total weight of the composition, of all silicone oils and non-silicone oils such as hydrocarbon oils, esters, and ethers within the composition.

(f) Carrier—As discussed above, the skin care compositions of the present invention may comprise a carrier. The first skin care composition may comprise a first carrier and the second skin care composition may comprise a second carrier wherein the first and second carriers provide a readily perceptible tactile difference. For example, the first skin care composition may comprise a first carrier in the form of a continuous aqueous phase emulsion and the second skin care composition may comprise a second carrier in the form of a continuous oil phase emulsion. The resulting tactile difference is that the first skin care composition may feel less oily or greasy compared to the other composition. In one embodiment, the first skin care composition comprises a first carrier in the form of a silicone-in-water emulsion and the second skin care composition comprises a second carrier in the form of a water-in-silicone emulsion.

(g) Tightening—The skin care compositions may provide a tightening sensation of the skin of a user. The tightening sensation may be provided by suitable firming agents. In a suitable embodiment, the first skin care composition comprises a firming agent and the second skin care composition is substantially free of a firming agent. The skin care composition may comprise a sufficient quantity of the firming agent to yield the tightening effect. In certain embodiments, the firming agents may be present in concentrations ranging from about 0.01% to about 40%, from about 0.1% to about 10%, or, alternatively, from about 1% to about 5% of the skin care composition.

Suitable firming agents include hydrolyzed proteins, partially-hydrolyzed proteins, and mixtures thereof. These proteins may be chemically modified with quaternary groups, fatty groups, fatty alkyl quaternary groups, silicone groups, or may be a protein copolymer. The source of the protein is not limited. Plant derived proteins include soya proteins, wheat proteins, almond protein, potato protein, oat proteins, pea proteins, sun flower proteins, corn proteins, cottonseed proteins, peanut proteins, and wheat germ protein. Other non-limiting examples include compounds containing hydrolyzed vegetable protein (and) hydrolyzed vegetable starch such as CROPEPTIDE W, hydrolyzed vegetable protein polysiloxane copolymers such as CRODASONE W, and hydrolyzed vegetable protein polyvinylpyrrolidone copolymers such as Hydrotriticum PVP; all available from Croda, Inc., Edison, N.J. Animal derived proteins include milk proteins, such as β-lactoglobulin, casein, or whey; serum proteins, such as horse serum; placental proteins; albumen; amylase; collagen; crystalline; cytochrome C; elastin; fibronectin; gelatin; gliadin; keratin; lipase; and serum albumin. Other suitable firming agents include polyurethane-14 and AMP-Acrylates copolymer mixture (such as the DynamX line from National Starch & Chemical Company, Bridgewater, N.J.), sodium polystyrene sulfonate (such as the Flexan line from National Starch), modified corn starch (such as the Amaze line from National Starch), polyimide-1 (such as AquaflexXL-30 from International Specialty Products, Wayne, N.J.), and vinylpyrrolidone/acrylates/lauryl methacrylate copolymer (such as Styleze 2000 from International Specialty Products).

(ii) Visible Differences—The personal care kit of the present invention comprises a first series of unit-dose packages containing a first skin care composition and a second series of unit-dose packages containing a second skin care composition wherein the first skin care composition and the second skin care composition exhibit a readily perceptible visible difference. The tactile difference may be any difference between the first skin care composition and the second skin care composition that is appreciable by the sense of sight (including color, shape, translucency, gloss, etc.). Exemplary tactile differences are provided below.

(a) Colorants—The first skin care composition may exhibit a color different than that of the second skin care composition. In one embodiment, the first skin care composition may comprise one or more colorants and the second skin care composition is substantially free of a colorant. The skin care composition may comprise a sufficient quantity of the colorant to yield the tightening effect. The present skin care composition from about 0.001% to about 25%, from about 0.01% to about 20%, or, alternatively, from about 0.1%% to about 15% of a colorant. In certain embodiments, the skin care composition is substantially free of a colorant when comprising less than 0.001%, less than about 0.0001%, or, alternatively, less than about 0.00001% of a colorant.

Suitable colorants may include inorganic or organic pigments and powders. Organic pigments can include natural colorants and synthetic monomeric and polymeric colorants. Organic pigments include various aromatic types such as azo, indigoid, triphenylmethane, anthraquinone, and xanthine dyes which are designated as D&C and FD&C blues, browns, greens, oranges, reds, yellows, etc. Organic pigments may consist of insoluble metallic salts of certified color additives, referred to as the Lakes. Inorganic pigments include iron oxides, ferric ammonium ferrocyanide, manganese violet, ultramarines, chromium, chromium hydroxide colors, and mixtures thereof. The pigments may be coated with one or more ingredients that cause the pigments to be hydrophobic. Suitable coating materials that will render the pigments more lipophilic in nature include silicones, lecithin, amino acids, phospholipids, inorganic and organic oils, polyethylene, and other polymeric materials. Suitable silicone treated pigments as disclosed in U.S. Pat. No. 5,143,722. Inorganic white or uncolored pigments include TiO2, ZnO, or ZrO2, which are commercially available from a number of sources. Other suitable colorants are identified in U.S. Pat. No. 7,166,279.

(b) Percent Transmission—The skin care compositions may be opaque (i.e., turbid) or translucent. The presence of colorants, insoluble particles, gas bubbles, large internal phase domains, and the like can affect the turbidity/translucency of a skin care composition. Opacity and translucency may be quantitatively determined by the percent transmission. Skin care compositions yielding a percent transmission of greater than or equal to 60% are considered translucent. Skin care composition yielding a percent transmission of less than 60%, 55%, 50%, 40%, or, alternatively, 30% are considered opaque. In one embodiment, the first skin care comprising is translucent and the second skin care composition is opaque.

Percent transmission involves measuring the transmission of light through a collagen film to which the skin care composition has been applied, relative to a control film. The methodology requires a light source sufficiently powerful for sample illumination, a camera and video frame grabber for capturing the image of the sample on the collagen surface and a computer with video imaging software for data analysis and for viewing on a video monitor. Suitable image capture and analysis software includes Optimas 5.2 from Optimas Corp., Washington (refer to Image Analysis Software guide Volume I), the software and guide being incorporated herein by reference.

Using a collagen film (such as IMS #1192 or equivalent available from IMS Inc., Milford, Conn.) mounted in a suitable holder, apply 40 microliters (using a Microman M50 pipette) of product and spread evenly by hand on the film surface using 10 finger rotations. Optionally, the sample is mounted on a Zeiss SV-11 microscope (or equivalent) equipped with a 1× lens (the microscope is useful for enlarging the image which the camera is capturing; the effective magnification of this system is about 5 microns/pixel). A mounting template can optionally be employed to aid in repositioning the sample for multiple measurements. The SV-11 should be set up so that maximum light is being transmitted to the camera (e.g., a Sony 760-MD CCD 3 Camera). To insure proper positioning and a clear image, the equipment is set in the following manner. Camera controls are set so that the Gamma and Linear matrix switches are off. The camera control box settings are further defined as follows: Gain=0, White/Black balance on auto, iris- auto, mode-camera, detail-12 o'clock position, phase-0 degrees, SC-3 o'clock position, H-12 o'clock position, Color temp −3200K, shutter off. The camera should be allowed to warm up for 15 minutes before adjusting white and black balance. Press the button labeled “white” to adjust the white balance, and adjust black balance by pulling the black adjustment rod and pushing the button that says black. Computer cables are connected to the RGB I and Composite Sync.ports on the camera. The microscope Iris is set to completely open and a frosted glass plate is positioned in the microscope base for a uniformly lit field. A clear glass plate may optionally be used to adjust the sample height. Open the Optimas 5.2 program on the computer. Use a sample cup which is partially covered with black tape in the light path to adjust gain and offset (brightness). The reflecting mirror at the base of the microscope is set for maximum reflection into the microscope. The mean of the light source should be 245 to 254.5. The STD Deviation should be less than 3. If the mean is out of specifications check the light bulb alignment and mirror adjustment.

For the control, an untreated piece of collagen film (such as IMS #1192 available from IMS Inc., Milford, Conn.), is mounted on a sample cup which is placed on the microscope such that the film lies in the center of the light path. The film is focused and light transmission through the film is measured using the image capture and analysis software. Multiple measurements are taken from separate areas of the sample, repositioning and refocusing the film for each measurement (seven or more measurements are taken). The histogram mean and standard deviation are determined using the image capture and analysis software.

For measuring light transmission by the test product, a piece of collagen film is first pre-hydrated with distilled water to insure flexibility. 40 microliters of test product is then dispensed on the film (e.g., using a Microman M50 pipette or equivalent), and spread evenly over its surface to produce an even film covering the surface of the collagen (generally by lightly spreading the material by applying 10 rotations of the finger, wearing a clean, latex finger cot, to the material). After waiting for a period of 5 minutes, the sample is mounted on the microscope base. Light transmission measurements through the film and product are then taken in the manner described for the control. Least significant differences can be performed on the data using Fischer's LSD method.

The coverage index and % transmission are calculated as follows:

$$Coverage Index = \frac{Control Mean - Test Product Mean}{Control Mean} \times 100$$

$$Percent Transmission = 100 - Coverage Index$$

(c) Matting Effect—A skin care composition can provide a matting effect (e.g., non-shiny) to the skin. The matting effect may help hide imperfections such as large pores, fine lines and wrinkles, and uneven skin tone while maintaining the natural appearance of the skin (e.g., without unacceptable skin whitening or discoloration). In one embodiment, the first skin care provides a matting effect and the second skin care composition does not.

The matting effect may be provided through the use of a requisite amount of silicone elastomer. In one embodiment, the first skin care composition comprises greater than about 1% of a silicone elastomer (on a solids basis as measured without carriers or other solvents) and the second skin care composition is substantially free of silicone elastomer. In other embodiments, the first skin care composition may comprise greater than about 2%, 3%, or, alternatively, 5% of a silicone elastomer. In certain embodiments, the second skin care composition may comprises less than about 0.1%, about 0.01%, or, alternatively, about 0.001% of a silicone elastomer.

Suitable silicone elastomers include crosslinked organopolysiloxane (or siloxane) elastomers, as described in U.S. patent publication 2003/0049212A1. The silicone elastomers may be emulsifying (e.g., having at least one polyoxyalkylene or polyglycerin moiety) or non-emulsifying silicone elastomers (e.g., essentially free of polyoxyalkylene or polyglycerin moieties).

Suitable non-emulsifying silicone elastomers include the CTFA (Cosmetic, Toiletry, and Fragrance Association International Cosmetic Ingredient Dictionary and Handbook, 11^{th} ed.) designated dimethicone/vinyl dimethicone crosspolymers such as supplied by Dow Corning™ (DC 9506), General Electric™ (SFE 839), Shin Etsu™ (KSG 15 and 16), and Grant Industries (GRANSIL RPS-NA) and dimethicone/phenyl vinyl dimethicone crosspolymer such as KSG 18 available from Shin Etsu™. Other exemplary silicone elastomers include the CTFA designated dimethicone crosspolymers including Dow Corning™ (DC 9040, DC 9041, DC 9045).

Suitable emulsifying silicone elastomers include CTFA designated dimethicone/PEG-10 crosspolymers such as KSG 24; dimethicone/PEG-10 crosspolymers such as KSG 21 and KSG 210; PEG-15/lauryl dimethicone crosspolymers such as KSG 31, KSG 32, KSG 33, KSG 310, KSG 320, KSG 330; PEG-15/lauryl dimethicone crosspolymers and PEG-10/lauryl dimethicone crosspolymers such as KSG 34 and KSG 340; dimethicone/polyglycerine-3 crosspolymers such as KSG-710; and lauryl dimethicone/polyglycerine-3 crosspolymers such as KSG 810, KSG 820, KSG 830, and KSG 840. All KSG materials are available from Shin Etsu™.

The matting effect may be provided through the use of a requisite amount of matting particles. In one embodiment, the first skin care composition comprises greater than about 3% of a matting particle and the second skin care composition is substantially free of silicone elastomer. In other embodiments, the first skin care composition may comprise greater than about 5%, about 10%, or, alternatively, about 15% of the matting particle. In certain embodiments, the second skin care composition may comprises less than about 1%, about 0.1%, or, alternatively, about 0.01% of the matting particles.

Matting particles have a refractive index of less than about 1.7. In other embodiments, the matting particles have a refractive index from about 1.3 to about 1.6 as this range closely matches the refractive index of skin. The matting particles may have an average particle size of from about 0.1 to about 40 μm. The average particle size is measured when the particulate material is in the neat form (i.e., in the essentially pure, powder form prior to combination with a carrier). Refractive index can be determined by conventional methods. The matting particles may have a substantially spherical shape or a non-platelet shape (i.e., no one dimension of the particle is at least 5-times smaller than the other two dimensions).

Suitable organic matting particles include those made of polymethylsilsesquioxane (e.g., Tospearl® 145 from GE Toshiba Silicone Co., Ltd., which has a median particle size of about 4.5 μm), polyamide (e.g., Orgasol® 2002 D NAT COS available from Elf Atochem SA), polythene, polyacrylonitrile, polyacrylic acid, polymethacrylic acid, polystyrene, polytetrafluoroethylene (PTFE) and poly(vinylidene chloride). Copolymers derived from monomers of the aforementioned materials can also be used (e.g., EA-209® from Kobo Products, Inc., which is an ethylene/acrylic acid copolymer having a median particle size of about 10 μm). Suitable inorganic matting particles include silica and boron nitride.

(d) Luminescent Effect—A skin care composition can provide a shine, glow, or a luminescent-effect. In one embodiment, the first skin care provides the shine, glow, or a luminescent-effect and the second skin care composition does not.

In one embodiment, the luminescent effect may be provided by a skin care composition having a requisite percentage of non-volatile oils. “Non-volatile,” as used herein, means a material that exhibit a vapor pressure of no more than about 0.2 mm Hg at 25° C. at one atmosphere and/or a material that has a boiling point at one atmosphere of at least about 300° C. In one embodiment, the first skin care composition comprises greater than 10% of non-volatile oils and the second skin care composition comprises less than 10% of non-volatile oils. In other embodiments, the first skin care composition may comprise greater than about 15%, about 20%, or, alternatively, about 25% of non-volatile oils and the second skin care composition comprises less than about 5%, about 1%, or, alternatively, about 0.1% of non-volatile oils.

Suitable non-volatile, polar oils include, but are not limited to, silicone oils, hydrocarbon oils; fatty alcohols, fatty acids, esters of mono and dibasic carboxylic acids with mono and polyhydric alcohols, polyoxyethylenes, polyoxypropylenes, polyoxyethylene and/or polyoxypropylene ethers of fatty alcohols, and mixtures thereof. Suitable non-volatile, non-polar oils include, but are not limited to, non-volatile polysiloxanes, paraffinic hydrocarbon oils, and mixtures thereof. Suitable polysiloxanes include polyalkylsiloxanes, polyarylsiloxanes, polyalkylarylsiloxanes, poly-ethersiloxane copolymers, and mixtures thereof. Exemplary polysiloxanes include Viscasil™ series (General Electric), Dow Corning 200 series (Dow Corning Corp.), SF 1075 methyl-phenyl fluid (General Electric), and 556 Cosmetic Grade Fluid (Dow Corning Corp.). Suitable non-volatile paraffinic hydrocarbon oils are described in U.S. Pat. No. 5,019,375 and in U.S. Publication No. 2003/0049212A1 and include mineral oils and branched-chain hydrocarbons such as Permethyl™ 102A, 103A and 104A (Permethyl Corporation), and Ethylflo™ 364 (Ethyl Corp.).

The luminescent effect may be provided by a skin care composition comprising platelet particles. The skin care composition may comprise a sufficient quantity of the platelet particles to yield the luminescent effect. In certain embodiments, the first skin care composition comprises at least about 2% of platelet particles and the second skin care composition is substantially free of platelet particle. In other embodiments, the first skin care composition may comprise at least about 5% of platelet particles. In one embodiment, second skin care composition comprises less than about 0.5%, 0.1%, or, alternatively, 0.01% of platelet particles.

Platelet particles are particles having one dimension that is at much smaller than in the other two dimensions. In certain embodiments, platelet particles have one dimension at least 5-times smaller than the other two dimensions and have a mean particle size of at least about 5 μm. The platelet shape allows the particle to provide specular reflection. Specular reflection refers to the phenomena where an incident beam of light is reflected at the equal and opposite angle to the angle of incidence. Platelets can for example be made of natural or synthetic mica, silica, alumina, glass, boron nitride, and bismuth oxychloride. As well these platelets can be coated with one or more layers of other ingredients, such as metal oxides (e.g., tin oxide, titanium dioxide, iron oxides), silica, and organic materials such as various silicones, soaps, etc. These coating may function to alter the optical properties of the material such as titanium dioxide coated micas that produce colored interference effects. The coatings may alter the polarity (e.g., hydrophilicity or hydrophobicity) of the particles. Further discussion of platelet particles is provided in U.S. Publication No. US2004/0234565A1.

(iii) Olfactory Differences—The personal care kit of the present invention comprises a first series of unit-dose packages containing a first skin care composition and a second series of unit-dose packages containing a second skin care composition wherein the first skin care composition and the second skin care composition exhibit a readily perceptible olfactory difference. The olfactory difference may be any difference between the first skin care composition and the second skin care composition that is appreciable by the sense of smell. In one suitable embodiment, the first skin care composition comprises a first fragrance and the second skin care composition is substantially fragrance free. In another embodiment, the first skin care composition comprises a first fragrance and the second skin care composition comprises a second fragrance different from the first.

D. Other Secondary Differences—While the present invention is directed, in part, to a personal care kit comprising a first skin care composition and a second skin care composition exhibiting a readily perceptible difference, other secondary differences may exist between the first and second skin care composition. In some embodiments, these secondary differences may be chronic differences that would not be readily appreciable to a user but may become appreciable over time. In other embodiments, the secondary differences may not become appreciable to a user.

In certain embodiments, the secondary difference may be a difference in the skin actives present within the skin care compositions. For example, a personal care kit may comprise a first skin care composition comprising a safe and effective amount (e.g., from about 1% to about 20%) of a sunscreen active and a second skin care composition substantially free of a sunscreen active. In another example, a personal care kit may comprise a first skin care composition comprising a skin active that may irritate the skin such as by having greater than about 0.05% of a retinoid and/or greater than 1% of a hydroxyacid and a second skin care composition substantially free of the skin active irritant.

In certain embodiments, the secondary difference may be a difference in the percent composition of a skin care active between skin care compositions. For example, a personal care kit may comprise a first skin care composition comprising a first weight percent of a skin active (e.g., 2% niacinamide) and a second skin care composition comprising a second weight percent (e.g., 1% or 4% niacinamide) of the skin care active, wherein the second weight percent is higher or lower than the first weight percent.

In certain embodiments, the secondary difference may be a difference in a device that is utilized during application. For example, a personal care kit may comprise a first series of unit-dose packages containing a first skin care composition that is applied with a first device and a second series of unit-doses packages containing a second skin care composition that is applied by hand or by a second device different from the first device. Suitable devices include microdermabrasion devices such as the Neutrogena Advanced Solutions At Home Microdermabrasion System.

### EXAMPLES

The following formulations are non-limiting examples of suitable skin care compositions that provide one or more of the above mentioned readily perceptible differences. Where applicable, ingredients are given in CTFA name. The skin care compositions are not specifically identified as a first or second skin care composition since a skilled artisan will readily recognize that various combinations of the Examples exist and may be used in the kit of the present invention. While particular embodiments of the subject invention have been described, it will be obvious to those skilled in the art that various changes and modifications to the subject invention can be made without departing from the spirit and scope of the invention. The Examples may be slightly modified by omitting key components that provide for the readily perceptible difference (e.g., Example 5 contains colorants FD&C Red 40 and Blue 1 which could be omitted to yield a colorant free examples). Specifically, one or more of components listed in boldface may be omitted to yield alternate examples.

| | (values are weight %) | | | | | | | | | |
|---|---|---|---|---|---|---|---|---|---|---|
| | Ex. 1 | Ex. 2 | Ex. 3 | Ex. 4 | Ex. 5 | Ex. 6 | Ex. 7 | Ex. 8 | Ex. 9 | Ex. 10 |
| PHASE A | | | | | | | | | | |
| DC-9040 *1 | 5.10 | 13.5 | | 50.7 | | | | | | |
| DC-9045 *2 | | | 15 | | 32.75 | 14.0 | | 14.0 | | |
| PEG-4 | | | | | | | | | q.s to | |
| | | | | | | | | | 100 | |
| Dimethicone | 4.10 | | 6 | | | 5.2 | | 5.2 | | |
| Polymethylsilsesquioxane *3 | 4.10 | 7.5 | | | | | 0.5 | | | 0.5 |
| Polyethylene beads *4 | | | | | | | | 2.0 | 2.0 | |
| Cyclomethicone | 11.40 | 23.5 | 15 | | 10.0 | 1.05 | | 1.05 | | |
| KSG-210 *5 | 5.40 | 2.5 | | | | | | | | |
| KSG-310 *6 | | | | 20.0 | | | | | | |
| Polyethylene wax *7 | 2.05 | | | | | | | | | |
| DC-2503 Cosmetic Wax *8 | 3.77 | | | | 1.5 | | | | | |
| Abil EM97 *9 | | 0.45 | | | 0.45 | | | | | |
| KF 6017 *10 | | 0.375 | | | | | | | | |
| Cetyl Ricinoleate | | 0.25 | | | | | | | | |
| KTZ Fine TiO₂ coated Mica *11 | 1.00 | | | | | | | | | |
| Dow Corning 1503 *12 | | | 3 | | | 3.5 | | 3.5 | | |
| Octisalate | | | | 4.0 | | | | | | |
| Homosalate | | | | 4.0 | | | | | | |
| Octocrylene | | | | 1.5 | | | | | | |
| Avobenzene | | | | 2.0 | | | | | | |
| Isopropyl Lauroylsarcosinate | | | | 7.5 | | | | | | |
| Tospearl 145A *13 | | | | 10 | | | | | | |
| Prestige Fire Red 11S2 *14 | | | | 0.1 | | | | | | |
| Microthene FN-510 *15 | | | 9 | | | 10.0 | | 10.0 | | |
| Petrolatum | | | | | 0.5 | | | | | |
| Isohexadecane | | | | | | | 3.0 | | | 3.0 |
| Isopropyl Isostearate | | | | | | | 1.0 | | | 1.0 |
| Stearic Acid | | | | | | | 0.4 | | | 0.4 |
| Cetearyl Glucoside | | | | | | | 0.2 | | 0.5 | 0.2 |
| Cetyl Alcohol | | | | | | | 1.0 | | 1.3 | 1.0 |
| Stearyl Alcohol | | | | | | | | | 20.0 | |
| Magnesium Sulfate Anhydrous | | | | | | | | | 3.0 | |
| PEG/PEG-300/55 Copolymer | | | | | | | | | 2.0 | |
| Tego Care CP *16 | | | | | | | | | 1.78 | |
| Econol TM-22 *17 | | | | | | | | | 0.80 | |
| Distearyldimonium chloride | | | | | | | | | 0.25 | |
| Hydroxypropylcellulose | | | | | | | | | | |
| Petrolatum | | | | | 0.5 | | | | 0.15 | |
| Fragrance | 0.10 | | | 0.2 | | | | | | |
| PHASE B | | | | | | | | | | |
| Glycerin | 10.00 | 10.00 | 11 | | 10 | 10.0 | 2.0 | 10.0 | | 2.0 |
| Panthenol | 0.5 | 1.00 | 0.7 | | 1.0 | 1.0 | | 1.0 | | |
| Pentylene Glycol | 3.00 | | | | | | | | | |
| Propylene Glycol | | 1.00 | | | 1.0 | 1.0 | | 1.0 | | |
| Butylene Glycol | | 1.00 | | | 1.0 | 1.0 | | 1.0 | | |
| Tocopherol Acetate | | 0.50 | 0.2 | | 0.5 | 0.5 | 0.5 | 0.5 | | 0.5 |
| N-Acetyl Glucosamine | 0.50 | | 2.0 | | | | | | | |
| Hexamidine Diisethanoate *18 | 0.10 | | | | | | | | | |
| Niacinamide | 5.00 | 4.00 | 5.00 | | 5 | 5.0 | 2.5 | 5.0 | | 2.5 |
| Methylparaben | 0.20 | 0.10 | | | 0.1 | | | | | |
| Ethylparaben | 0.05 | 0.10 | | | 0.1 | 0.1 | | 0.1 | | |
| Benzyl Alcohol | 0.25 | 0.50 | | | 0.4 | 0.4 | | 0.4 | 0.1 | |
| Propyl Paraben | | 0.10 | | | | 0.1 | | 0.1 | | |
| Disodium EDTA | | 0.10 | 0.05 | | 0.1 | | 0.1 | | | 0.1 |
| Polysorbate 20 | | | 0.6 | | | 0.8 | | 0.8 | | |
| Glydant Plus Liquid *19 | | | 0.3 | | | | | | | |
| Laureth-4 | | | 0.2 | | | 0.2 | | 0.2 | | |
| Sucrose Polycottonseedate | | | | | 0.5 | | | | | |
| Allantoin | | | | | 0.1 | 0.2 | | 0.2 | | |
| Prodew 400 *20 | | | | | | | | | | |
| GLW75CAP-MP *21 | | | | | | 0.35 | | 0.35 | | |
| Hydrolyzed wheat protein | | | | | | | 2.0 | | | |
| Menthol | | | | | | | | | | 0.5 |
| Vanillyl alcohol isoamyl ether | | | | | | | | | | 0.05 |
| monophosphate | | | | | | | | | | |
| Sodium Chloride | 0.50 | | | | | | | | | |
| FD&C Red No. 40 | | | | | .00025 | | | | | |
| FD&C Blue 1 | | | | | .00011 | | | | | |
| Water | q.s to | q.s to | q.s to | | q.s to | q.s to | q.s to | q.s to | | q.s to |
| | 100 | 100 | 100 | | 100 | 100 | 100 | 100 | | 100 |
| Thickener | | | | | | | | | | |
| Sepigel 305 *22 | | | 1.6 | | | 1.5 | 1.5 | 1.5 | | 1.5 |

*1 12.5% Dimethicone Crosspolymer in Cyclopentasiloxane. Available from Dow Corning.
*2 Dimethicone Crosspolymer in Cyclopentasiloxane. Available from Dow Corning.
*3 E.g., Tospearl 145A or Tospearl 2000. Available from GE Toshiba Silicone
*4 PFM (250-500 μm) colored beads from Kobo.
*5 25% Dimethicone PEG-10/15 Crosspolymer in Dimethicone. Available from Shin-Etsu
*6 PEG-15/Lauryl Dimethicone Crosspolymer in Mineral Oil from Shin-Etsu.
*7 Jeenate 3H polyethylene wax. Available from Jeen
*8 Stearyl Dimethicone. Available from Dow Corning.
*9 Bis-PEG/PPG-14/14 Dimethicone. Available from Degussa
*10 PEG-10 Dimethicone. Available from Shin-Etsu.
*11 Hydrophobically modified TiO₂ coated Mica. Available from Kobo.
*12 Dimethicone/Dimethiconol blend from Dow Corning.
*13 Polymethylsilsesquioxane from General Electric.
*14 Mica and iron oxides from Eckart.
*15 Polyethylene powder available from Equistar.
*16 Dioleoylethyl hydroxyethylmonium methosulfate mixture available from Degussa Care & Surface Specialties, Hopewell, VA.
*17 Behenyltrimethylammonium chloride in carrier available from Sanyo Performance Chemicals, JP.
*18 Hexamidine diisethionate, availabile from Laboratoires Serobiologiques.
*19 DMDM Hydrantoin and Iodopropynyl Butylcarbamate blend available from Lonza, Inc.
*20 Available from Ajinomoto U.S.A., Inc., Paramus NJ.
*21 TiO₂ with water, glycerine, polyacrylate, and methylparaben available from Kobo Products.
*22 Polyacrylamide, C13-14 Isoparaffin, and Laureth-7 blend from Seppic.

For example 1, combine the ingredients of Phase A in a suitable container. In a separate suitable container, combine the ingredients of Phase B. Heat each phase to 75-80° C. while mixing each phase using a suitable mixer (e.g., Anchor blade, propeller blade) until each reaches temperature and is homogenous. Slowly add Phase B to Phase A while continuing to mix Phase A. Continue mixing until batch is uniform. Homogenize product with Ultra-Turrax homogenizer (IKA, Inc) or equivalent and pour product into suitable containers at 75-80° C. Store the containers at room temperature without disturbing for at least 12 hours.

For examples 2 and 5, in a suitable container, combine the ingredients of Phase A and mix with a suitable mixer until homogenous. In a separate container, combine the ingredients of Phase B and mix until homogenous. Slowly add Phase B to Phase A while continuing to mix Phase A. Continue mixing until batch is uniform. Homogenize product with Ultra-Turrax homogenizer (IKA, Inc) or equivalent and pour product into suitable containers.

For examples 3, 6-8, and 10, in a suitable vessel, the water phase ingredients are combined and mixed until uniform; the water phase may be warmed to dissolve all ingredients. In a separate suitable container, the silicone/oil phase ingredients are combined and mixed until uniform; the silicone/oil phase may be warmed to dissolve all ingredients. Half the thickener and then the silicone/oil phase is added to the water phase and the resulting emulsion is milled (e.g., with a Tekmar T-25). The remainder of the thickener and then the remaining ingredients are then added to the emulsion while stirring. Once the composition is uniform, the product is poured into suitable containers.

For examples 4 and 9, in a suitable vessel, the ingredients are combined and mixed until uniform; the composition may be warmed to dissolve all ingredients. Once the composition is uniform, the product is poured into suitable containers.

A suitable unit-dose package for all of the examples may be a unidose plastic tube with tear-open tamper-proof closure available from CEBAL Tubes Europe.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care kit comprising:
a) a first series of unit-dose packages containing a first skin care composition,
b) a second series of unit-doses packages containing a second skin care composition, wherein the first skin care composition and the second skin care composition exhibit a readily perceptible difference.

2. The personal care kit of claim 1 wherein the perceptible difference is a visible difference.

3. The personal care kit of claim 2 wherein the visible difference is provided by the first skin care composition comprising at least about 0.001% of colorant and the second skin care compositions being substantially free of colorant.

4. The personal care kit of claim 2 wherein the visible difference is provided by the first skin care composition comprising at least about 1% of a platelet powder and the second skin care composition being substantially free of a platelet powder.

5. The personal care kit of claim 2 wherein the visible difference is provided by the first skin care composition comprising at least 0.3% of a powder having a refractive index of greater than about 2 and the second skin care composition being substantially free of a powder having a refractive index of greater than about 2.

6. The personal care kit of claim 2 wherein the visible difference is provided by the first skin care composition comprising at least about 0.5% of a powder having a refractive index of less than about 1.8 and the second skin care composition being substantially free of a powder having a refractive index of less than about 1.8.

7. The personal care kit of claim 2 wherein the visible difference is provided by the first skin care composition exhibiting a percent transmission of greater than 60% and the second skin care composition exhibiting a percent transmission of less than 60%.

8. The personal care kit of claim 2 wherein the visible difference is provided by the first skin care composition comprising at least about 2% of a silicone elastomer and the second skin care composition being substantially free of silicone elastomer.

9. The personal care kit of claim 1 wherein the perceptible difference is a tactile difference.

10. The personal care kit of claim 9 wherein the tactile difference is provided by the first skin care composition having a viscosity of less than 40,000 centipoise and the second skin care composition having a viscosity of more than 40,000 centipoise.

11. The personal care kit of claim 10 wherein the first skin care composition has a viscosity of less than about 30,000 centipoise and the second skin care composition has a viscosity of more than about 50,000 centipoise.

12. The personal care kit of claim 9 wherein the tactile difference is provided by the first skin care composition results in a post-application skin temperature change of at least 1° C. and the second skin care composition results in a post-application skin temperature change of no more than 0.25° C.

13. The personal care kit of claim 9 wherein the tactile difference is provided by the first skin care composition comprising a temperature sensate and the second skin care composition being substantially free of a temperature sensate.

14. The personal care kit of claim 9 wherein the tactile difference is provided by the first skin care composition comprising a warming temperature sensate and the second skin care composition comprising a cooling temperature sensate.

15. The personal care kit of claim 9 wherein the tactile difference is provided by the first skin care composition comprising an abrasive particulate material having an average particle size of greater than about 100 μm and the second skin care composition being substantially free of an abrasive particulate material having an average particle size of greater than about 100 μm.

16. The personal care kit of claim 9 wherein the tactile difference is provided by the first skin care composition comprising greater than 30% of one or more oils and the second skin care composition comprising no more than 30% of one or more oils.

17. The personal care kit of claim 16 wherein the tactile difference is provided by the first skin care composition comprising greater than about 40% of one or more oils and the second skin care composition comprising no more than about 20% of one or more oils.

18. The personal care kit of claim 9 wherein the tactile difference is provided by the first skin care composition comprising a first carrier in the form of an aqueous continuous emulsion and the second skin care composition comprising a second carrier in the form of an oil continuous emulsion.

19. The personal care kit of claim 18 wherein the aqueous continuous emulsion of the first carrier is a silicone-in-water emulsion and the oil continuous emulsion of the second carrier is a water-in-silicone emulsion.

20. The personal care kit of claim 1 wherein the perceptible difference is an olfactory difference.

21. The personal care kit of claim 20 wherein the olfactory difference is provided by the first skin care composition comprising a fragrance and the second skin care composition being substantially fragrance free.

22. The personal care kit of claim 20 wherein the olfactory difference is provided by the first skin care composition comprising a first fragrance and the second skin care composition comprising a second fragrance different from the first fragrance.

23. The personal care kit of claim 1 wherein the first skin care composition comprises at least one skin care active which the second skin care composition does not comprise.

24. The personal care kit of claim 23 wherein the skin care active is selected from a group consisting of sunscreens, retinoids, hydroxyacids, vitamins, peptides, sugar amines, oil control agents, tanning actives, anti-acne actives, desquamation actives, anti-cellulite actives, chelating agents, skin lightening agents, flavonoids, protease inhibitors, non-vitamin antioxidants and radical scavengers, hair growth regulators, anti-wrinkle actives, anti-atrophy actives, minerals, phytosterols and/or plant hormones, tyrosinase inhibitors, anti-inflammatory agents, N-acyl amino acid compounds, antimicrobials, antifungals, or mixtures thereof.

25. The personal care kit of claim 1 wherein the first skin care composition and the second skin care composition both comprise a skin care active, wherein the first skin care composition and the second skin care composition differ in the weight percent of the skin care active.

26. The personal care kit of claim 1 wherein the first series of unit-dose packages comprise a first use indicium and the second series of unit-doses packages comprise a second use indicium.

27. The personal care kit of claim 1 wherein the first series of unit-dose packages contain less than about 5 mL of the first skin care composition and the second series of unit-dose packages contain less than about 5 mL of the second skin care composition.

28. The personal care kit of claim 1 wherein the first series of unit-dose packages contain less than about 2 mL of the first skin care composition and the second series of unit-dose packages contain less than about 2 mL of the second skin care composition.

29. A personal care kit comprising:
a) a first series of unit-dose packages having a first use marking, each unit-dose package comprising less than about 5 mL of a first skin care composition, said first skin care composition comprising a aqueous continuous phase and at least one skin care active,
b) a second series of unit-doses packages having a second use marking, each unit-dose package comprising less than about 5 mL of a second skin care composition, said second skin care composition comprising a oily continuous phase and at least one skin care active.

30. A method for treating the skin comprising the steps of
a) dispensing and applying a first skin care composition from a first unit-dose package;
b) repeating step a) for a plurality of times at a first interval,
c) dispensing and applying a second skin care composition from a second unit-dose package,
d) repeating step d) at a second routine interval
wherein step c) begins after the plurality of times and wherein the first skin care composition and the second skin care composition exhibit a readily perceptible difference.

31. A method for treating the skin comprising the steps of
a) dispensing and applying a first skin care composition from a first unit-dose package once a day for m number of days,
b) dispensing and applying a second skin care composition from a second unit-dose package once a day for n number of days,
wherein step b) begins about 24 hours after the m^{th} day and wherein the first skin care composition and the second skin care composition exhibit a readily perceptible difference.

32. The method of claim 31 wherein m is at least 7 and n is at least 7.
